(19) United States
(12) Patent Application Publication
(10) Pub. No.: US 2025/0308631 A1
(43) Pub. Date: Oct. 2, 2025

(54) RESIDUALS METHOD TO DECOUPLE CORRELATED PHENOTYPES

(71) Applicant: Heritable Agriculture Inc., Mountain View, CA (US)

(72) Inventors: Ramon Viñas Torné, Bern (CH); Mathias Voges, San Francisco, CA (US); Gavin Duggan, Mountain View, CA (US); Bradley Zamft, Mountain View, CA (US); James Schnable

(73) Assignee: Heritable Agriculture Inc., Mountain View, CA (US)

(21) Appl. No.: 19/096,148

(22) Filed: Mar. 31, 2025

Related U.S. Application Data
(60) Provisional application No. 63/572,543, filed on Apr. 1, 2024.

(51) Int. Cl. G16B 20/50; G16B 25/10; G16B 40/20

(52) U.S. Cl. CPC G16B 20/50; G16B 25/10; G16B 40/20

(57) ABSTRACT

The present disclosure relates to techniques for decoupling correlated phenotypes and identifying driver genes of a target phenotype. The techniques include obtaining phenotype data gene expression profiles for samples. The phenotype data is input into a prediction model configured to learn relationships between the one or more other phenotypes and the target phenotype and predict measurements for the target phenotype. Residuals are determined between the predicted measurements for the target phenotype and the obtained measurements for the target phenotype, and used to label the gene expression profiles to train a machine learning model to predict residuals of the target phenotype and select the driver genes.

### CROSS-REFERENCES TO RELATED APPLICATION

The present application claims priority and benefit from U.S. Provisional Application No. 63/572,543, filed Apr. 1, 2024, the entire contents of which are incorporated herein by reference for all purposes.

### FIELD

The present disclosure relates to techniques for decoupling correlated phenotypes, and in particular, to leveraging prediction modeling and machine learning techniques as mechanisms for identifying genes that drive a target phenotype but no other correlated phenotypes in order to provide recommendations for ideal genes, their gene expression profiles, and the requisite genome edits, that are conducive to a desired target phenotype.

### BACKGROUND

In genetics, phenotype refers to a set of observable characteristics or traits of an organism. Phenotype is the result of two basic factors: the expression of the organism's genetic code (e.g., DNA or genotype) and the influence of environmental factors. Importantly, how the gene and environment interact can have a drastic effect on how a phenotype may be portrayed. For example, a plant may express the genes to promote growth or high leaf production, but if the plant is deprived of water or sunlight, it is unlikely to display either phenotype to its full potential.

With respect to the influence of genetics on phenotype, FIG. 1A illustrates how the transcription of genes encoded in DNA (e.g., the genome) into RNA (e.g., the transcriptome) and the translation of mRNA molecules into proteins (e.g., proteome) generate a set of small-molecule metabolites (e.g., metabolome) whose combined expression results in a set of all the traits expressed (e.g., phenome) of an organism. To determine which gene or sets of genes are responsible for a particular phenotype, researchers use methods known as forward and reverse genetic screenings. A forward genetic screen involves incorporating random mutations (in both location and mutation type) using mutagens (e.g., chemical compounds and irradiation). Experimentation is then used to determine which gene or genes were mutated that caused the change in phenotype. In reverse genetic screenings, genes are specifically targeted, and the impact of phenotype is observed. Through the implementation of these genetic methods, researchers have determined the function of many genes in various model organisms.

Despite these seemingly straightforward screening approaches, determining what genotype results in a particular phenotype can be incredibly challenging, especially when dealing with complex or polygenic traits (e.g., traits impacted by more than one gene) and different inheritance patterns (e.g., autosomal dominant/recessive, X-linked dominant/recessive, mitochondrial, codominance, incomplete dominance, mosaicism, epistasis, germline, somatic, etc.). The challenge becomes even greater when phenotypes are correlated, as it is difficult to identify genes that affect only one phenotype without influencing the other, particularly in cases of pleiotropy, where genes impact multiple traits. For example, in Arabidopsis, two correlated traits-leaf number and days to flowering-illustrate this issue (FIG. 1B): plants that flower later tend to have more leaves because they have more time to grow, and the more leaves an Arabidopsis plant has, the more delayed its flowering time. The goal is to disentangle genetic variation driving one phenotype from the correlation with the other. Addressing this challenge could have significant implications for climate change; for instance, diversity panels of corn have revealed differences in dry root biomass of up to 175 g—a fivefold increase. When scaled globally across all corn plants, this could lead to the sequestration of 1 gigaton (GT) of carbon annually, offering a substantial contribution to reducing global warming.

### SUMMARY

In various embodiments, a computer-implemented method is provided that comprises: obtaining phenotype data and gene expression profiles for samples, wherein the phenotype data comprises measurements for correlated phenotypes, and wherein the correlated phenotypes comprise a target phenotype and one or more other phenotypes; generating, using a prediction model, predicted measurements for the target phenotype for the samples based on relationships or correlations learned from the phenotype data for the one or more other phenotypes and the target phenotype; determining residuals between the predicted measurements for the target phenotype and the obtained measurements for the target phenotype; labeling, using the determined residuals, the gene expression profiles; training, using the labeled gene expression profiles, a machine learning model to predict residuals from the labeled gene expression profiles; and outputting the trained machine learning model.

In some embodiments, the prediction model is selected from a plurality of prediction models, each prediction model in the plurality of prediction models is configured to model linear relationships, nonlinear relationships, or any combination thereof; and the prediction model is configured to (i) model linear relationships and uses statistical functions to generate the predicted measurements for the target phenotype, (ii) model nonlinear relationships and uses statistical functions or machine learning models to generate the predicted measurements for the target phenotype, (iii) model both linear and nonlinear relationships and uses machine learning models to generate the predicted measurements for the target phenotype, or (iv) any combination of (i), (ii), and (iii).

In some embodiments, the training comprises: iterative operations to find a set of parameters for the machine learning model that minimizes a loss function for the machine learning model, wherein each iteration includes finding the set of parameters for the machine learning model so that a value of the loss function using the set of parameters is smaller than a value of the loss function using another set of parameters in a previous iteration, and wherein the loss function is configured to measure a difference in the predicted residuals and the determined residuals.

In some embodiments, the machine learning model is selected from a plurality of machine learning models, and wherein each machine learning model in the plurality of machine learning models can model either linear relationships, non-linear relationships, or any combination thereof.

In some embodiments, the machine learning model includes: (i) high interpretability and low accuracy that models linear relationships, (ii) low interpretability and high accuracy that models nonlinear relationships, (iii) high interpretability and high accuracy that models linear relationships, non-linear relationships, or any combination thereof, or (iv) any combination of (i), (ii), and (iii).

In some embodiments, the training comprises performing permutation testing, which comprises: (a) shuffling the determined residuals in order to re-label the gene expression profiles; (b) training the machine learning model, using the re-labeled gene expression profiles, to predict permuted residuals for the target phenotype; (c) repeating (a) and (b) for a sufficient number of permutations to create an approximate test statistic null-distribution; and (d) determining, based on the approximate test statistic null-distribution, statistical scores for each feature in the gene expression profiles.

In various embodiments, a computer-implemented method is provided, comprising: accessing a transcriptomic dataset for a set of correlated phenotypes comprising a target phenotype and one or more other phenotypes; inputting the transcriptomic dataset into a machine learning model constructed for a task of predicting a residual that represents variation of the target phenotype that cannot be explained by the one or more other phenotypes; generating, using the machine learning model, a predicted residual for the target phenotype based on the transcriptomic dataset; analyzing decisions made by the machine learning model to predict the residual, wherein the analyzing comprises: generating (i) feature importance scores or (ii) statistical scores for features used in the prediction of the residual, and ranking or otherwise sorting the features based on the feature importance score or the statistical scores associated with each of the features; identifying, a set of candidate genes for the target phenotype as having a largest contribution or influence on the residual based on the analyzing; and identifying, based on the set of candidate genes, a set of genomic regions that when edited provides a requisite change in a gene expression profile to realize an expected phenotypic change.

In some embodiments, the transcriptomic dataset is collected from a sample, and wherein the transcriptomic dataset comprises expression data for all the genes in the sample or for a subset of genes in the sample.

In some embodiments, the machine learning model is configured to model linear relationships and wherein the feature importance scores are directly identified.

In some embodiments, the machine learning model utilizes one or more non-linear relationships to generate the predicted residual and wherein analyzing decisions comprises using an explainable artificial intelligence system and wherein the feature importance scores are indirectly identified using the explainable artificial intelligence system.

In some embodiments, the statistical scores are generated by performing permutation testing to create an approximate test statistic null-distribution.

In some embodiments, a system is provided that includes one or more data processors and a non-transitory computer readable medium storing instructions which, when executed on the one or more data processors, cause the one or more data processors to perform part or all of one or more methods or processes disclosed herein.

In some embodiments, a computer-program product is provided that is tangibly embodied in a non-transitory machine-readable medium and that includes instructions configured to cause one or more data processors to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the subject matter claimed. Thus, it should be understood that although the present application has been specifically disclosed by embodiments and optional features, modification and variation of the concepts herein disclosed may be resorted to by those skilled in the art, and that such modifications and variations are considered to be within the scope of this application as defined by the appended claims.

### BRIEF DESCRIPTION OF THE DRAWINGS

The present application will be better understood in view of the following non-limiting figures, in which:

FIG. 1A shows a diagram illustrating a central dogma theory for how gene expression impacts phenotypes in accordance with various embodiments.

FIG. 1B shows a diagram illustrating certain plant phenotypes are correlated in accordance with various embodiments.

FIG. 2 shows a block diagram illustrating a system for implementing techniques to train a machine learning model to predict residuals in accordance with various embodiments.

FIG. 3 shows a flowchart illustrating a process for training a machine learning model to decouple two or more phenotypes in accordance with various embodiments.

FIG. 4 shows a flowchart illustrating a process for identifying candidate genes using a trained machine learning model in accordance with various embodiments.

FIG. 5 shows a block diagram of a gene discovery and editing system in accordance with various embodiments.

FIGS. 6A-6C are scatter plots illustrating the relationship between two correlated phenotypes in accordance with various embodiments.

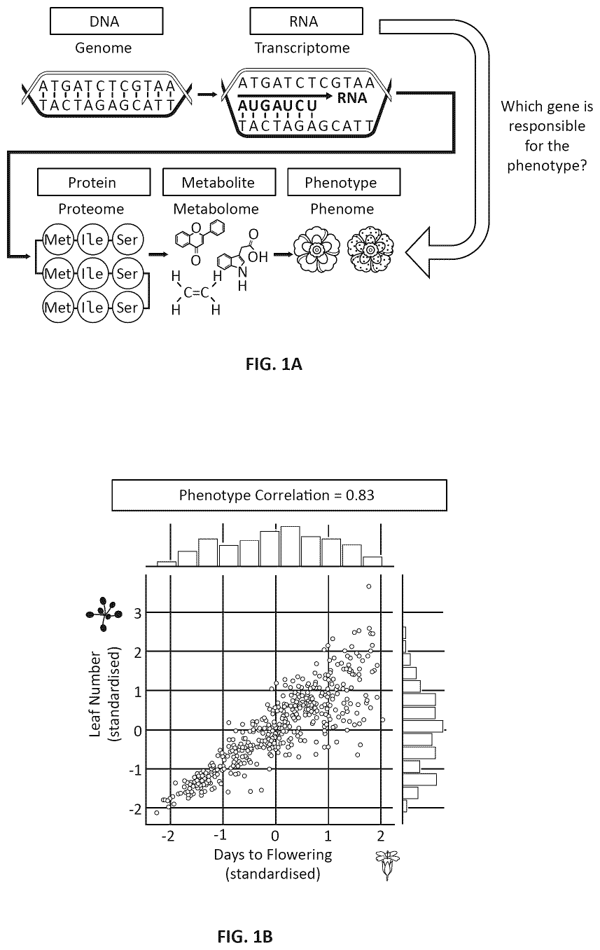

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed but could have additional steps not included in a figure. A process may correspond to a method, a function, a procedure, a subroutine, a subprogram, etc. When a process corresponds to a function, its termination may correspond to a return of the function to the calling function or the main function.

### I. INTRODUCTION

Plant genetic engineering methods have evolved from traditional practices that rely on natural genetic variation via evolutionary forces (e.g., selection, mutation, migration, genetic drift, etc.) to select for favorable genetic changes to more advanced practices of targeted genetic engineering using genetic tools (e.g., zinc finger nucleases (ZFNs), transcription activator-like effector nucleases (TALENs) and CRISPR-Cas9). Regardless of the method used, the end goal of both these practices is to introduce genetic variability that produces desirable characteristics in the plant/crop. Examples of this may include high yield potential, large seeds, drought resistance, pest and disease resistance, photosensitivity, etc. In addition to improving overall crop production, the impact of plant genetic engineering on the environment and climate change is also of great importance, particularly in how plant engineering could help mitigate climate change.

Plants play a fundamental role in the recycling of carbon (e.g., the carbon cycle). The carbon cycle is part of a biochemical cycle by which the carbon in the atmosphere (in the form of carbon dioxide (CO₂) gas) is absorbed by plants for photosynthesis (e.g., carbon sequestration) and then released back into the atmosphere as plants decompose. CO₂ enters the atmosphere through processes such as respiration and the burning of fossil fuels. Atmospheric CO₂ helps to balance energy (e.g., heat from the sun) to keep the planet warm enough to support life. However, increased levels of atmospheric CO₂ are a major contributing factor to global warming in addition to deforestation, overpopulation, and excessive release of fossil fuels. As a consequence of these practices, the carbon cycle has become grossly imbalanced.

To combat this, plant engineers are exploring genetic opportunities to design plants/crops that are more environmentally friendly. One example of this is to genetically engineer crops with larger root biomass, given that most crops only have a root biomass that extends about 1 meter below ground. As plants absorb CO₂, they take the carbon into their root system and further down into the soil. In fact, soil has been observed to sequester twice as much carbon as the atmosphere, thus creating crops with desirable below ground carbon sequestration traits is highly desirable. To demonstrate this point, attempts have been made to engineer some varieties of corn to produce 5-fold larger root biomass. Considering corn is the fourth largest crop by market size, with 40 trillion plants grown in the world per year, a 5-fold increase in root biomass equates to the sequestration of upwards of 1 gigaton of carbon per year and a dramatic effect on climate change. In addition, a higher root biomass would also have positive benefits on soil health and drought resistance to boot. However, in crops such as corn, the root biomass phenotype is negatively correlated with the plant yield phenotype. In other words, an increase in root biomass leads to a decrease in plant yield, which can adversely affect farmers who rely on plant production for their livelihood. Despite having the methodology and infrastructure to target specific genes contributing to a desired phenotype, it often comes at the expense of losing control over other correlated traits. Typically, correlated phenotypes share a significant overlap in genes and those genes that are phenotype-specific (i.e., impact one phenotype but not the other correlated phenotypes) are not well characterized.

To address these limitations and problems, a machine learning pipeline is disclosed herein that predicts a target phenotype from phenotype measurement data and generates phenotype-specific residuals that are used to identify phenotype-specific genes. The machine learning pipeline can be broken down into three components. The first component predicts a target phenotype based on phenotype measurement data collected from samples, using a phenotype prediction model selected from a plurality of phenotype prediction models. More specifically, the plurality of phenotype prediction models comprises models configured to identify or learn linear relationships, nonlinear relationships, or any combination thereof between a target phenotype and one or more correlated phenotypes. A phenotype-specific residual can then be determined by subtracting the predicted target phenotype (e.g., output phenotype measurement from the prediction model) from the one or more correlated phenotypes (e.g., obtained phenotype measurement data). The residual represents phenotype-specific variations that cannot be explained by the one or more correlated phenotypes. The second component in the machine learning pipeline uses the phenotype-specific residuals to label gene expression profiles collected from the same samples where the phenotype measurement data is collected from. The labeled gene expression profiles are then used by the third component in the machine learning pipeline to predict residuals. A machine learning model of the third component learns the relationships or correlations between the genes in the gene expression profiles that result in the labeled residual. Accordingly, the genes that contribute the most to the residual are identified, either directly for linear relationships or indirectly (e.g., via explainable AI) for nonlinear relationships. The identified genes can be experimentally tested through genetic manipulations or editing to confirm that they only affect the target phenotype without impacting other correlated phenotypes.

In one exemplary embodiment, a computer-implemented method is provided that comprises obtaining phenotype data and gene expression profiles for samples, wherein the samples have correlated phenotypes, the correlated phenotypes comprise one or more other phenotypes and a target phenotype, and the phenotype data comprises measurements for the correlated phenotypes. Relationships or correlations are learnt from the phenotype data for the one or more other phenotypes and the target phenotype using a prediction model, and predicted measurements for the target phenotype for the samples are generated using the prediction model. Residuals are then determined between the predicted measurements for the target phenotype and the obtained measurements for the target phenotype and used to label the gene expression profiles. The labeled gene expression profiles are then used to train a machine learning model to predict residuals from the labeled gene expression profiles and the trained machine learning model is output for downstream applications. The output may further include a set of candidate genes for the target phenotype as having a largest contribution or influence on the residual based on an analysis of the training and features of the machine learning model.

The disclosed techniques are capable of identifying driver genes that specifically contribute to the development of the target phenotype, while minimizing or eliminating influence on other related phenotypes. By leveraging machine learning models, these techniques isolate genetic factors directly linked to the desired phenotype, ensuring precision in distinguishing primary contributors from secondary or unrelated effects. This approach enhances the reliability of phenotype-specific gene identification, reducing noise and improve the accuracy of gene identification, enabling targeted interventions to derive plants with the target phenotype, offering a distinct advantage over conventional methods and enabling more effective therapeutic or research applications.

### II. MACHINE LEARNING PIPELINE

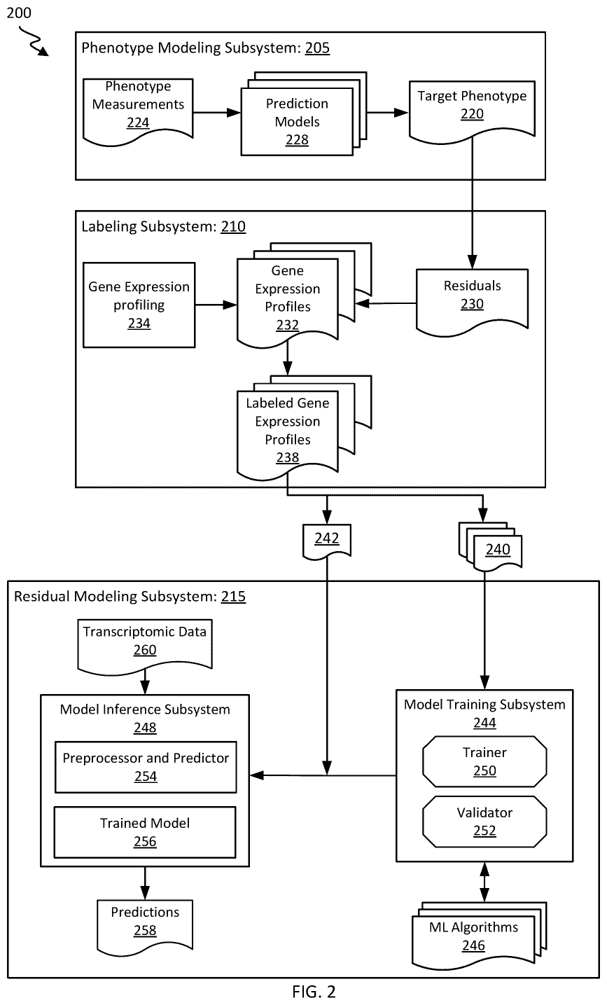

FIG. 2 shows a block diagram for a machine learning pipeline 200 comprising three subsystems that work together to predict a target phenotype and generate labels to be used by a machine learning model to predict phenotype-specific residuals in accordance with various embodiments. The machine learning pipeline 200 comprises a phenotype modeling subsystem 205 for predicting target phenotypes, a labeling subsystem 210 for labeling gene expression profiles from samples with ground truth residuals, and a residual modeling subsystem 215 for predicting phenotype-specific residuals from the labeled gene expression profiles. The machine learning pipeline 200 can be implemented in hardware and/or software systems using physical components and computational frameworks. For example, the machine learning pipeline 200 can leverage cloud-based servers equipped with high-performance GPUs, TPUs, or CPUs for computationally intensive tasks, local hardware workstations with high-speed memory and specialized processors for secure and efficient data handling, and edge devices for real-time deployment of machine learning models. By integrating these hardware components with software frameworks (e.g., TensorFlow or PyTorch), the machine learning pipeline 200 can achieve scalability, adaptability, and efficiency, enabling seamless deployment across diverse research and clinical environments.

The phenotype modeling subsystem 205 comprises a plurality of prediction models 228 configured to predict a target phenotype 220 as a function of other phenotype measurements 224. Phenotype refers to observable characteristics of a sample that are influenced by gene expression and environmental factors. As described herein, “sample” refers to any plant, including all related genus, species, and genetically altered plants known in the art. A non-limiting and non-exhaustive list of exemplary plants include Arabidopsis thaliana, Boechera genus, Selaginella moellendorffii, Brachypodium distachyon, Setaria viridis, Lotus japonicus, Lemna gibba, Medicago truncatula, Mimulus guttatus, Nicotiana benthamiana, Nicotiana tabacum BY-2, Populus, Chlamydomonas reinhardtii, Physcomitrella patens, and Marchantia polymorpha. In addition, agricultural plants may also be included, for example: Zea mays (corn), Oryze sativa (rice), Triticum aestivum (wheat), Medicago sativa (alfalfa), Hordeum vulgare (barley), Glycine max (soybean), etc. In other instances, “sample” may refer to any organism known in the art, including modified or genetically modified organisms that have multiple correlated phenotypes. In these instances, “sample” may refer to prokaryotes such as bacteria, or eukaryotes such as fungi and animals, or viruses or other non-cellular organisms.

In some instances, genetic variation and/or different environmental stimuli may alter phenotypes across samples even if their genetic background is the same. Phenotype measurements 224 are often used to document these changes in desired phenotypes such as height, root biomass, bud density, leaf shape, color, fruit or grain production, drought/insect resistances, and the like. More specifically, the phenotype measurements 224 can be any scalar value (height, mass, volume, count, age, etc.) descriptive of any desired phenotype and represent expected target phenotypes. For example, phenotype measurements for Arabidopsis thaliana could include plant height (e.g., 15 cm), root biomass (e.g., 0.8 g), bud density (e.g., 30 buds per plant), and leaf color (e.g., quantified via RGB values). These measurements can serve as scalar values descriptive of target phenotypes and represent actual or expected outcomes. Data structures such as dictionaries, arrays, tuples, matrices, tables, graphs, databases, or pandas DataFrames can be employed to effectively store the phenotype measurements.

A prediction model can be selected from a plurality of prediction models 228 that are configured to identify or learn relationships between the phenotype measurements 224 of correlated phenotypes to predict the target phenotype 220. Correlated phenotypes, or phenotypic correlation, describes samples with high phenotype measurements for one phenotype (e.g., target phenotype 220) while also tending to have high (or low) phenotype measurements for another phenotype (e.g., one or more other phenotypes). In some instances, the relationship between the target phenotype 220 and the one or more other phenotypes is linear, and a prediction model 228 configured to model linear relationships (such as simple linear regression, multiple linear regression, polynomial regression, ElasticNet regression, and the like) is used. For example, one prediction model 228 may use a simple linear regression that involves fitting a linear equation (y=mx+b) to the phenotype measurements 224, where one phenotype (y) is predicted as a function of the other (x) phenotype(s).

As used herein, the term “high” refers to phenotype measurements (e.g., a trait such as height) that are greater than a threshold, indicating an elevated level or presence of a particular phenotype relative to other phenotype measurements. Conversely, the term “low” refers to phenotype measurements that are less than the threshold, indicating a reduced level or presence of a particular phenotype relative to other phenotype measurements. In some embodiments, the threshold is predetermined or determined using established using statistical or domain-specific methods. For example, a “high” phenotype measurement might be defined as values above the 75th percentile (upper quartile) of a population, and a “low” phenotype measurement might be defined as values below the 25th percentile (lower quartile). In another example, “high” means values greater than one or two standard deviations above the mean, and “low” means values less than one or two standard deviations below the mean. In some embodiments, machine learning techniques, such as clustering algorithms (e.g., k-means or hierarchical clustering), can be used to group phenotype measurements into “high” and “low” categories based on inherent patterns in the population data. In some embodiments, the threshold is determined by one or more expert in the field.

In other instances, the relationship between the target phenotype 220 and the one or more other phenotypes is nonlinear or any combination of both linear and nonlinear relationships and more powerful predictive models such as machine learning algorithms including neural networks (e.g., Deep Neural Network (DNN)), decision trees (e.g., CART (Classification and Regression Trees)), or EBM (explainable boosting machine) models are used. Nonlinear relationships often occur when one or more of the correlated phenotypes is a binary, ordinal (discrete traits controlled by multiple genes), or continuous phenotype. Neural networks model both linear and nonlinear relationships using activation functions. Neural networks without activation functions are essentially just linear regression models, regardless of the number of layers. The activation functions are mathematical functions (e.g., sigmoid, Hyperbolic Tangent (tanh), Rectified Linear Unit (ReLU), Leaky ReLU, Softmax, and the like) applied to the output of each neuron in a neural network, specifically in the context of deep learning. They introduce nonlinearity to the network, allowing it to learn and model complex relationships between inputs and outputs (e.g., between the phenotype measurements 224 for a target phenotype and one or more other phenotypes), which is important for modeling the more complex correlation problems described herein. The nonlinearity is introduced by transforming the output of each neuron, allowing the network to learn complex patterns in the input data. The type of activation function used may be dictated by the type of phenotype being targeted or explored. For example, to model binary phenotypes, a sigmoid activation function may be used. For ordinal or multiclass phenotypes, a softmax function may be used and for continuous phenotypes, a hyperbolic tangent or linear functions may be used.

In the context of traditional decision trees, activation functions as seen in neural networks are not used to introduce nonlinearity. Decision trees use a different approach to make splits at nodes based on feature thresholds that minimize impurity (for classification) or reduce variance (for regression). While each individual decision in a decision tree is linear in nature (i.e., a comparison based on a threshold for a specific feature), the combination of multiple decision points in a tree structure allows decision trees to represent nonlinear relationships between features and the target variable (e.g., a target phenotype 220). By recursively splitting the data based on different features and their thresholds, decision trees create complex, nonlinear boundaries between classes or predict nonlinear relationships in regression problems. This ability to partition the feature space into regions of different classes or regression values enables decision trees to handle nonlinear relationships effectively. Moreover, ensemble methods such as Random Forests or Gradient Boosting, which combine multiple decision trees, are particularly adept at capturing nonlinear patterns in the data. These ensembles improve the modeling of complex relationships by aggregating the predictions of multiple decision trees, making them more powerful in capturing nonlinearities than individual trees.

With regards to EBM models, these models are a tree-based, cyclic gradient boosting generalized additive model with automatic interaction detection. EBM models are “glass box” models meaning they are configured to have high interpretability as well as accuracy that is just as high as “black-box” neural networks. High accuracy is achieved because EBM learns each feature function using machine learning techniques (e.g., bagging or boosting) in a cycle fashion, where each cycle, the model learns one feature at a time. In doing so, the EBM learns the best feature function for each feature and thus can show how each feature contributes to the model's prediction. In addition, the automatic detection and inclusion of pairwise interaction terms also contributes to the high accuracy performance. The high interpretability of the model is attributed to its additive effect, meaning that each feature contributes to the predictions in a modular way making interpretations regarding feature contribution easy to understand and therefore easy to visualize by plotting the feature function.

Once a relationship is identified or learned, the prediction model 228 uses the relationship to generate a predicted measurement for the target phenotype 220. For example, a linear relationship may be identified between two correlated phenotypes (e.g., root biomass and corn production), wherein for every 4 units of root biomass gained, grain production reduced by 1 unit (i.e., m=−0.25 in a linear equation where y=grain production (target phenotype) and x=root biomass). The phenotype prediction model will use m=−0.25 and phenotype measurements for root biomass to predict measurements for grain production. This allows for the residuals 230, representing phenotype-specific variations that are not accounted for by other correlated phenotypes, to be determined. To determine the residuals 230, the predicted measurements for the target phenotype are subtracted from the observed phenotype measurements 224 collected from the samples. The residuals 230 are used as ground truth labels in labeling subsystem 210.

Labeling subsystem 210 comprises gene expression profiles 232 obtained from the same samples where the phenotype measurements 224 are collected from. Gene expression is the process by which the information encoded in a gene is turned into a function (e.g., the transcription of a gene into mRNA molecules that code for proteins). In other words, phenotype is a reflection of all the proteins (i.e., the proteome) expressed in a cell. To measure the levels of gene expression in a sample, gene expression profiling techniques 234 are used to measure the amount of mRNA molecules expressed in cells at any given moment. Examples can include microarray analysis, reverse transcription polymerase chain reaction (RT-PCR), or RNA-sequencing. Gene expression profiling techniques 234 output the gene expression profiles 232 that comprise expression data for all the genes in a sample at the particular moment in time the sample was collected. If a gene is being expressed by the sample (i.e., being transcribed into mRNA), the gene is considered ‘on’ within the gene expression profiles 232; and if the gene is not being expressed by the sample, the gene is considered ‘off’ within the gene expression profiles 232.

In some instances, the gene expression profiles 232 are transformed into a set of numerical representations of gene expression (e.g., log-transformed, standardized, or 0-1 scaled gene expression profiles). Further, additional transformations may be done to account for the impact of environmental and maintenance conditions on gene expression. Environmental conditions include location-specific environmental conditions the plant is exposed to, e.g., temperature, precipitation, soil properties, and the like. Maintenance conditions include any adjustable aspect of the management of the growth of the plant, e.g., inputs such as fertilizer or water, the timing of planting, fertilizing, harvesting, and the like.

As described above, the labeling subsystem 210 uses the residuals 230 to label the gene expression profiles 232 and generate a dataset of labeled gene expression profiles 238. In some instances, the dataset may be a transformed version (e.g., log-transformed or standardized gene expression profiles) with ground truths. Further, the dataset of labeled gene expression profiles 238 may be split into training and validation datasets 240 as well as a testing dataset 242. The splitting may be performed randomly (e.g., 70% training, 15% validating, and 15% testing) or the splitting may be performed in accordance with a more complex validation technique such as K-Fold Cross-Validation, Leave-one-out Cross-Validation, Leave-one-group-out Cross-Validation, Nested Cross-Validation, or the like to minimize sampling bias and overfitting. The training portion of the data is used to train the machine learning model to learn the learnable parameters (e.g., weights and biases), while the validating portion is used for tuning hyper-parameters and selecting the optimal non-learnable parameters (e.g., parameters that are not updated during training). The testing portion of the data represents data the machine learning model has never seen before in order to estimate the general performance of the model.

Once the labeled gene expression profiles 238 are split, they enter the residual modeling subsystem 215 for predicting phenotype-specific residuals. The residual modeling subsystem 215 includes the model training subsystem 244 for training and validating a machine learning model and the model inference subsystem 248 for testing and using the machine learning model in an inference phase. The model training subsystem 244 comprises two systems: a trainer 250 and a validator 252 for training and validating machine learning algorithms 246 to be used by the other subsystems, such as the model inference subsystem 248 for a given task (e.g., predicting residuals from transcriptomic data).

Although not explicitly shown, the residual modeling subsystem 215 can store a plurality of different machine learning models capable of modeling linear relationships, nonlinear relationships, or any combination thereof. In some instances, a machine learning model with high interpretability and low accuracy is used to model linear and smooth relationships (e.g., k-nearest neighbors, decision trees, linear regression, classification rules). Such a machine learning model may be selected if the relationship identified by the prediction model 228 in the phenotype modeling subsystem 205 is also linear. In other instances, a machine learning model with low interpretability and high accuracy is used to model linear or nonlinear relationships or any combination thereof (e.g., deep neural networks (DNN), graph neural network (GNN), or support vector machine). In other instances, combinations of the abovementioned modeling approaches may also be used. Such a machine learning model may be selected if the relationship learned by the prediction model 228 in the phenotype modeling subsystem 205 is linear or nonlinear. In further instances, a machine learning model with high interpretability and high accuracy (e.g., EBM model) is used to model linear or nonlinear relationships or any combination thereof. It should be understood that the teachings herein are applicable to machine learning models that model either linear relationships, non-linear relationships, or any combination thereof.

Trainer 250 and validator 252 are part of a machine learning operationalization framework comprising hardware such as one or more processors (e.g., a CPU, GPU, TPU, FPGA, the like, or any combination thereof), memory, and storage that operates software or computer program instructions (e.g., TensorFlow, PyTorch, Keras, and the like) to execute arithmetic, logic, input and output commands for the machine learning model. More specifically, trainer 250 performs iterative operations of training that involve inputting portions of training data into machine learning algorithms 246 to find a set of model parameters (e.g., weights and/or biases) that minimize objective functions (e.g., loss/error function, cost function, modified cross entropy loss, etc.). The objective function can be constructed to measure the difference between the outputs inferred using the models (e.g., predicted residuals) and the ground truth (e.g., determined residuals 230) annotated to the samples using the labels. For example, for a supervised learning-based model, the goal of the training is to learn a function “h( )” (also sometimes referred to as the hypothesis function) that maps the training input space X to the target value space Y, h: X→Y, such that h(x) is a good predictor for the corresponding value of Y. Various different techniques may be used to learn this hypothesis function. In some machine learning algorithms, such as neural networks, this is done using back propagation. The current error is typically propagated backwards to a previous layer, where it is used to modify the weights and biases in such a way that the error is minimized. The weights are modified using the optimization function. Optimization functions usually calculate the error gradient (i.e., the partial derivative of the objective function with respect to the weights) and the weights are modified in the opposite direction of the calculated error gradient. For example, techniques, such as back propagation, random feedback, Direct Feedback Alignment (DFA), Indirect Feedback Alignment (IFA), Hebbian learning, and the like, are used to update the model parameters in such a manner as to minimize this objective function. This cycle is repeated until the minimum of the objective function is reached.

Trainer 250 also performs the process of selecting hyperparameters, using an optimization algorithm, to find the model parameters that correspond to the best fit between prediction and actual outputs. Example optimization algorithms include a stochastic gradient descent algorithm or a variant thereof such as batch gradient descent or minibatch gradient descent. The hyperparameters are settings that can be tuned or optimized to control the behavior of the machine learning algorithms 246. Most models explicitly define hyperparameters that control different aspects of the models such as memory or cost of execution. However, additional hyperparameters may be defined to adapt a model to a specific scenario. For example, the hyperparameters may include the number of hidden units of a model, the learning rate of a model, the convolution kernel width, the number of kernels for a model, the number of graph connections to make during a lookback period, the maximum depth of a tree in a random forest, a minimum sample split, a maximum number of leaf nodes, a minimum number of leaf nodes, and the like.

Once a set of model parameters are identified, the model has been trained and is then validated using the validation datasets 240 by validator 252. The validation process includes iterative operations of inputting the validating datasets 240 into the machine learning algorithms 246 using a validation technique such as K-Fold Cross-Validation, Leave-one-out Cross-Validation, Leave-one-group-out Cross-Validation, Nested Cross-Validation, or the like to fine tune the hyperparameters and ultimately find the optimal set of hyperparameters. Once the optimal set of hyperparameters are obtained, a reserved set of the testing dataset 242, from the initial splitting of the labeled gene expression profiles 238, are input into trained model 256 to obtain output (in this example, predicted residuals describing the target phenotype-specific variation that cannot be explained by the one or more other phenotypes that correlate with the target phenotype), and the output is evaluated versus ground truth values (e.g., the residuals 230 determined by the phenotype modeling subsystem 205) using correlation techniques such as Bland-Altman method and the Spearman's rank correlation coefficients and calculating performance metrics such as the error, accuracy, precision, recall, receiver operating characteristic curve (ROC), etc.

The model training subsystem 244 outputs a trained model 256 with an optimized set of model parameters and hyperparameters for use in the model inference subsystem 248. The model inference subsystem 248 generates inference phase predictions 258 provided to users using a preprocessor and predictor 254 and the trained model 256. For example, the preprocessor and predictor 254 executes processes for inputting transcriptomic data 260 (e.g., gene expression profiles from samples) into a trained model 256. Then the trained model 256 will output predictions 258 (e.g., residuals describing the target phenotype-specific variation that cannot be explained by the one or more other phenotypes, candidate driver genes for the target phenotype, or the like).

The preprocessor and predictor 254 are part of the machine learning operationalization framework comprising hardware such as one or more processors (e.g., a CPU, GPU, TPU, FPGA, the like, or any combination thereof), memory, and storage that operates software or computer program instructions (e.g., Application Programming Interfaces (APIs), Cloud Infrastructure, Kubernetes, Docker, TensorFlow, Kuberflow, Torchserve, and the like) to execute arithmetic, logic, input and output commands for executing a machine learning model in a production environment. In some instances, the preprocessor and predictor 254 implement deployment of the model using a cloud platform such as Amazon Web Services (AWS), Google Cloud Platform (GCP), and Microsoft Azure. A cloud platform makes machine learning more accessible, flexible, and cost-effective while allowing developers to build and deploy the model faster.

### III. DECOUPLING TWO OR MORE PHENOTYPES USING COMPUTATIONAL TECHNIQUES

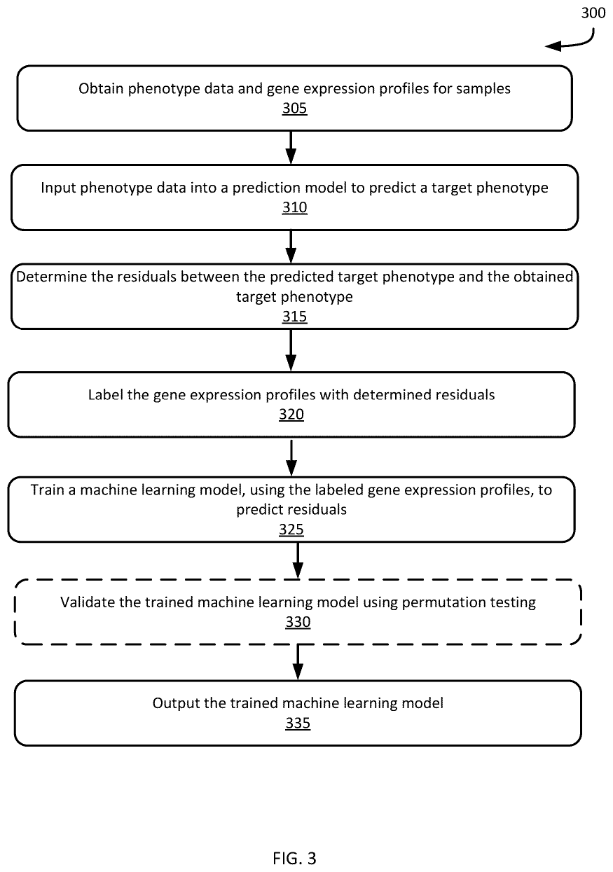

FIG. 3 is a flowchart illustrating a process 300 for training a machine learning model to decouple two or more phenotypes in accordance with various embodiments. The process 300 depicted in FIG. 3 may be implemented in software (e.g., code, instructions, program) executed by one or more processing units (e.g., processors, cores) of the respective systems, hardware, or combinations thereof (e.g., the intelligent selection machine). The software may be stored on a non-transitory storage medium (e.g., on a memory device). The method presented in FIG. 3 and described below is intended to be illustrative and non-limiting. Although FIG. 3 depicts the various processing steps occurring in a particular sequence or order, this is not intended to be limiting. In certain alternative embodiments, the steps may be performed in some different orders, or some steps may also be performed in parallel. In some embodiments, such as the embodiments depicted in FIG. 2, the process 300 depicted in FIG. 3 may be performed by the components of the machine learning pipeline 200 described with respect to FIG. 2.

Process 300 begins at block 305 where phenotype data and gene expression profiles are obtained from samples. Each phenotype measurement can be a scalar value (e.g., height, mass, volume, count, age, etc.) describing a phenotype of a sample. In some instances, the phenotype data includes measurements from correlated phenotypes including a target phenotype and one or more other phenotypes. Correlated phenotypes, or phenotypic correlation, describes samples with high phenotype measurements for one phenotype (e.g., target phenotype) while also tending to have high (or low) phenotype measurements for another phenotype (e.g., one or more other phenotypes). The phenotype data may represent observed phenotype measurements, or expected phenotype measurements based on the corresponding gene expression profiles obtained from the same samples. For example, given the sample specific gene expression profile, expected phenotype measurements may include: 60 days to flowering, 30 leaves, 175 grams root biomass, 8 corn stalks, etc. In some embodiments, the phenotype data is stored in a specific data structure such as dictionaries, arrays, tuples, matrices, tables, hierarchical clustering trees, graphs, databases, or pandas DataFrames.

The gene expression profiles may include quantitative measurements of gene activity, such as RNA transcripts or protein levels, that provide insight into the biological processes governing the phenotypes or phenotypic traits. Each sample-specific gene expression profile can include thousands of data points representing expression levels for multiple genes. These profiles may be to identify genes or gene networks that influence correlated phenotypes, such as flowering time, leaf production, or biomass, and select candidate driver genes that influence the specific target phenotype but not other correlated phenotypes. For example, a gene expression profile might reveal elevated transcription levels for genes associated with photosynthesis and growth, correlating with observed phenotypes like rapid flowering (60 days), increased leaf count (30 leaves), or enhanced root biomass (175 grams). To store and analyze gene expression profiles efficiently, data structures such as dictionaries, arrays, tuples, matrices, tables, hierarchical clustering trees, graphs, databases, or pandas DataFrames can be employed to capture the high-dimensional data, enabling sophisticated statistical and machine learning models to predict expected phenotype measurements or residuals based on gene activity patterns.

At block 310, the phenotype data is input into a prediction model (e.g., one of the prediction models 228 described with respect to FIG. 2). The prediction model can be constructed for a task of predicting a measurement for a target phenotype as output data based on measurements for the one or more other phenotypes by an algorithm learning relationships or correlations between the one or more other phenotypes and the target phenotype. The prediction model may be selected from a plurality of prediction models, where each prediction model in the plurality of prediction models is configured to model linear relationships, nonlinear relationships, or any combination thereof. To determine the type of relationship, the phenotype data for the target phenotype and the one or more other phenotypes may be input into each of the plurality of prediction models to determine which model is the best fit (e.g., determining the model that most effectively captures the underlying patterns or relationships within the data, leading to accurate predictions or representations). The best fit may be determined based on one or more factors including accuracy/performance, generalization, simplicity, interpretability, computational efficiency, robustness, specific domain considerations, and the like. Determining the best fit model often involves a trade-off between these factors. Techniques such as cross-validation, hyperparameter tuning, and rigorous evaluation on test data can be used in making an informed decision about the best fit model for a given task such as predicting a target phenotype. The plurality of prediction models may include, without limitation: linear statistical functions (e.g., linear regression or multiple regression), nonlinear statistical functions (e.g., quadratic, exponential, cubic, hyperbolic, sigmoid, etc.), highly interpretable machine learning models (e.g., k-nearest neighbors, decision trees, random forest, linear regression, classification rules, EBM), or machine learning models with low interpretability (e.g., deep neural networks (DNN), graph neural network (GNN), or support vector machine).

In some instances, the relationship between the target phenotype and the one or more other phenotypes is linear. For example, one prediction model configured to model a simple linear regression (y=mx+b) may be used to predict the target phenotype (y) as a function of another correlated phenotype (x). In some instances, the relationship between the target phenotype and the one or more other phenotypes is nonlinear, for example ordinal phenotypes that are controlled by multiple genes. A different prediction model configured to use a softmax activation function may instead be used. Regardless of the prediction model used, a predicted measurement for the target phenotype is generated based on the relationships or correlations learned from the phenotype data for the one or more other phenotypes and the target phenotype.

At block 315, the residuals between the predicted measurements for the target phenotype and the obtained (observed, or expected) measurements for the target phenotype are determined. Residuals in the context of regression analysis represent the differences between the observed values and the predicted values from a model and may be calculated by subtracting the predicted values (as a vector) from the actual observed values (as another vector). For example, if the subsystem is presented with a set of observed values yᵢ and the corresponding predicted values ŷᵢ from the regression model. The residuals, denoted as eᵢ, can be calculated as follows:
a. For Each Data Point: Calculate the residual for each data point i by subtracting the predicted value from the observed value:

$${e}_{i} = {y}_{i} - \hat{{y}_{ι}} \qquad ( 1 )$$

b. Vector Form: Collect all these individual residuals into a vector e, which represents the residuals for the entire dataset:

$$e = [ \begin{matrix}{e}_{1} \\ {e}_{2} \\ ⋮ \\ {e}_{n}\end{matrix} ] \qquad ( 2 )$$

Where n represents the number of data points in the dataset, and e is a vector containing all the residuals for each data point. The residuals in the present context represent phenotype-specific variations of the target phenotype that cannot be explained by the one or more other correlated phenotypes.

At block 320, the gene expression profiles are labeled with the determined residuals from block 315. A sample-specific gene expression profile comprises expression data for some or all the genes in a sample at a particular moment in time when the sample was collected. In some cases, expression data from all genes was collected, and only a subset of the expression data is used. The gene expression profiles may be transformed in a way that takes into account the impact of environmental (e.g., temperature, precipitation, etc.) and maintenance conditions (e.g., timing of planting, harvesting, etc.) on the gene expression.

At block 325, the labeled gene expression profiles are used to train a machine learning model to predict residuals. Training includes performing iterative operations to find a set of parameters for the machine learning model that minimize a loss function. Each training iteration finds an updated set of parameters that decreases the value of the loss function compared to a set of parameters used in a previous iteration. By minimizing the loss function, the machine learning model continues to reduce its performance error associated with each round of training. In other words, the machine learning model learns to minimize the difference between the predicted residuals and the determined residuals.

The machine learning model used to predict residuals is based on a machine learning algorithm selected from a plurality of machine learning algorithms, where each machine learning algorithm in the plurality of machine learning algorithms can model linear relationships, non-linear relationships, or both. In some instances, the choice of machine learning algorithm is determined based on the relationship identified or learned by the prediction model at block 310. Moreover, the labeled gene expression profiles may be input into each of the plurality of machine learning algorithms to determine which model performs the best. In some cases, a machine learning algorithm with high interpretability and low accuracy that models linear relationships proves to be the best algorithm. In other instances, a machine learning algorithm with low interpretability and high accuracy that models either linear, nonlinear relationships, or any combination thereof performs better. In further instances, a machine learning algorithm with high interpretability and high accuracy that models either linear, nonlinear relationships, or any combination thereof performs better.

At block 330, training may also comprise validating the trained machine learning model by performing permutation testing. The permutation testing may be performed using the labeled gene expression profiles to predict the residuals of a target phenotype. Permutation testing is a type of statistical test that is designed to measure the effect of some treatment(s) on experimental units (e.g., the effect of one or more correlated phenotypes on another phenotype). The goal is to test whether there is a difference between the control residuals (ground truth labels determined at block 315) and the predicted residuals output from the trained machine learning model. Prior to input into the machine learning algorithm, the ground truth residuals are randomly shuffled and used to re-label the gene expression profiles. This breaks a true association between the gene expression profiles and the residuals, simulating a null hypothesis. The re-labeled gene expression profiles are input into the machine learning algorithm with using the same hyperparameters, training process, and evaluation procedure as for the trained machine learning model at block 325, and the machine learning algorithm will predict the permuted residual. Training will iterate, using different permutations (e.g., shuffled residuals) until a sufficient number of permutations have been completed to create an approximate test statistic null-distribution.

The approximate test statistic null-distribution approximates all the possible test statistics that may be observed, assuming there is no difference between the predicted and control residuals (e.g., the null hypothesis). The approximate test statistic may be compared to statistic of the trained machine learning model at block 325 to calculate a p-value. If the p-value is small (e.g., p<0.05), the observed performance is unlikely to have occurred by chance, suggesting that the training machine learning model captures a meaningful relationship between gene expression and residuals.

Permutation testing not only validates the robustness of machine learning models but also provides actionable insights into the biological relevance of specific features. Candidate driver genes for a target phenotype (e.g., drought resistance or flowering time) can be identified by ranking genes based on their contribution to the model's predictions. For instance, if a particular gene consistently achieves a high importance score with a statistically significant false discovery rate (FDR), it can be prioritized as a candidate for targeted genetic modification, further functional studies, or other downstream applications. Specifically, the approximate test statistic null-distribution generated during permutation testing can be used to calculate the FDR for each feature or gene, quantifying the likelihood that its assigned importance score reflects a true association rather than random noise.

Beyond determining individual driver genes, permutation testing enables the determination of gene networks and pathways that collaboratively influence phenotypes, offering a comprehensive view of the underlying biological mechanisms. Gene networks represent interconnected groups of genes that work together to regulate biological processes, while pathways describe sequences of biochemical events and interactions that drive cellular functions. The process of detecting gene networks may begin with analyzing correlations between gene expression levels across samples. Highly correlated genes are likely to belong to the same functional network or pathway, as their activities are often synchronized due to shared regulatory mechanisms or involvement in similar biological processes. The correlated genes may be grouped into clusters using statistical or machine learning techniques, such as hierarchical clustering or other clustering algorithms, which help define the structure of gene networks. These networks can be visualized as graphs, where nodes represent genes and edges represent interactions or correlations between them, offering a clear picture of how genes work together to influence phenotypes.

Once gene networks are identified, pathway enrichment analysis can be performed to determine if the clusters of genes are associated with known biological pathways. This involves comparing the identified gene sets against pathway databases, such as KEGG (Kyoto Encyclopedia of Genes and Genomes), Reactome, or Gene Ontology (GO). For example, a network of genes with high importance scores for drought resistance may be enriched in the abscisic acid (ABA) signaling pathway, a pathway in plant responses to water stress. Another approach for identifying gene networks involves co-expression analysis, which groups genes into co-expression modules based on their expression patterns across samples. These modules often correspond to biological pathways or functional groups, making them highly relevant for understanding the mechanisms driving phenotypes. To validate and expand the identified networks, cross-reference them with gene interaction databases such as STRING or BioGRID may be performed. These gene networks and pathways behave may also be analyzed or validated under different conditions or over time. For instance, dynamic pathway analysis can reveal how gene expression and network interactions shift in response to environmental stimuli, such as drought, heat, or nutrient availability. This temporal or condition-specific analysis provides deeper insights into the adaptability and regulation of biological systems.

The term “sufficient number” is defined as any number necessary to build a test statistic distribution that includes or approximates all possible test statistic values that can be observed under a null hypothesis. For example, the sufficient number can be about 100, 500, 1000, 5000, 10000, or 20000 times. In some instances, the sufficient number needed for a permutation test depends on the desired statistical precision and computational constraints. Permutation testing can be computationally expensive, particularly for large datasets or complex models. The sufficient number can be determined by balancing the need for precision with available computational resources. In some instances, a test statistic for every possible permutation is determined to build an exact distribution. In other instances, a statistically significant sample size of test statistic values is determined to build an approximate distribution.

Finally, at block 335, training has concluded, and the trained machine learning model is output that predicts a phenotype-specific residual from gene expression profiles. In some embodiments, the output also includes the sets of candidate driver genes. The output may be provided to a machine learning operationalization framework comprising hardware such as one or more processors (e.g., a CPU, GPU, TPU, FPGA, the like, or any combination thereof), memory, and storage that operates deployment tools including software or computer program instructions (e.g., Application Programming Interfaces (APIs), Cloud Infrastructure, Kubernetes, Docker, TensorFlow, Kuberflow, Torchserve, and the like) to execute arithmetic, logic, input and output commands for executing the trained machine learning model in a production environment. In some instances, the deployment tools implement deployment of the trained machine learning model using a cloud platform such as Amazon Web Services (AWS), Google Cloud Platform (GCP), and Microsoft Azure. A cloud platform makes machine learning more accessible, flexible, and cost-effective while allowing developers to build and deploy the fine-tuned decoder model faster.

### IV. IDENTIFICATION OF THE GENES THAT CONTRIBUTE TO A TARGET PHENOTYPE

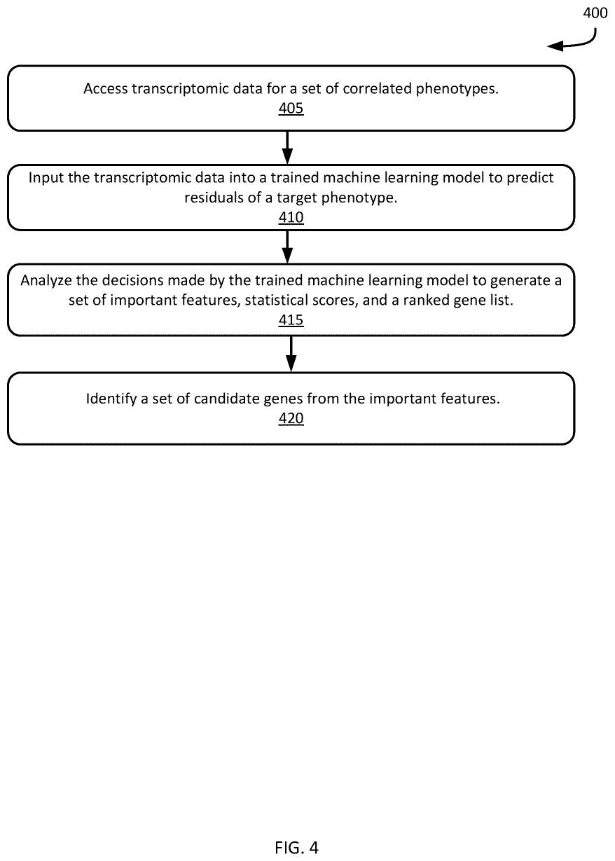

FIG. 4 is a flowchart illustrating a process 400 for how a trained machine learning model, which has been trained to predict phenotype-specific residuals (e.g., same residuals described in FIGS. 2 and 3) using transcriptomic data (e.g., gene expression profiles described in FIGS. 2 and 3), identifies phenotype-specific features that contribute to a target phenotype, but not other correlated phenotypes. In other words, given a specific transcriptomic or gene expression profile, the machine learning model is trained to learn the relationships between the features that result in the predicted phenotype-specific residuals. The process 400 depicted in FIG. 4 may be implemented in software (e.g., code, instructions, program) executed by one or more processing units (e.g., processors, cores) of the respective systems, hardware, or combinations thereof (e.g., the intelligent selection machine). The software may be stored on a non-transitory storage medium (e.g., on a memory device). The process 400 presented in FIG. 4 and described below is intended to be illustrative and non-limiting. Although FIG. 4 depicts the various processing steps occurring in a particular sequence or order, this is not intended to be limiting.

Starting at block 405, transcriptomic data from samples are acquired for a set of correlated phenotypes comprising a target phenotype and one or more other correlated phenotypes. The transcriptomic data may comprise expression data for all the genes in a sample. In some instances, the transcriptomic data may comprise expression data for a subset of the genes in the sample.

At block 410, the transcriptomic data is input into a trained machine learning model to predict residuals of a target phenotype that represents variations of the target phenotype that cannot be explained by the one or more other correlated phenotypes. The trained machine learning model processes the transcriptomic data set by extracting relevant features that contribute to the predicted residual. Processing involves using the appropriate computational operations based on the machine learning model selected from the plurality of machine learning models and applying the learned patterns realized through model parameters to process the extracted relevant features and calculate the predictions. For example, if the trained machine learning model used has high interpretability and low accuracy (e.g., k-nearest neighbors, decision trees, linear regression, classification rules) computational operations specific to those algorithms are used. In other instances, if the trained machine learning model used has low interpretability and high accuracy (e.g., deep neural networks (DNN), graph neural network (GNN), random forest, or support vector machine) computational operations specific to those algorithms are used. In further instances, the trained machine learning model used as high interpretability and high accuracy (e.g., EBM model) computational operations specific to those algorithms are used. Regardless of the type, the trained machine learning model will generate a predicted residual for the target phenotype based on the transcriptomic dataset.

At block 415, the decisions made by the trained machine learning model are analyzed. Analyzing comprises (i) generating a set of feature importance scores for features (e.g., genes from the transcriptomic data) used in the prediction of the residual, (ii) generating statistical scores for features used in the prediction of the residual, and (iii) ranking or otherwise sorting the features based on the feature importance score or the statistical scores associate with each of the features. How the feature importance scores are generated for the features used in the prediction of the residual is dependent on the type of machine learning model used. Important features are directly identified from trained models with high interpretability. For example, if the machine learning model is a linear regression model, the standardized regression coefficient (also referred to as the beta coefficient or beta weight) for each feature used in the model would function as a feature importance score describing the relative magnitude of the effect of different features. Additionally or alternatively, statistical scores may be generated and assigned to the features used to predict the residual by performing permutation testing as described with respect to FIG. 3. A significant statistical score indicates that the feature importance score (e.g., contribution to the predicted residual) is in fact statistically different, and thus a true result. Once each feature has a feature importance score and/or a statistical score assigned to it, the features are ranked or otherwise sorted by either score. On the other hand, if a trained machine learning model with low interpretability is used, an explainable artificial intelligence (XAI) is used to determine how the model determined its predicted residual and what information (e.g., features) were used to reach that prediction. Then feature importance scores, statistical scores, and a ranked gene list are obtained.

XAI refers to techniques in the application of artificial intelligence (AI) such that the decisions (e.g., the solution results) of a machine learning model can be understood and interpreted. XAI contrasts with the concept of the “black box” in machine learning where even the designers of a machine learning model cannot explain why the AI arrived at a specific decision by the machine learning model. Examples of techniques used for XAI include, without limitation, SHapley Additive exPlanations (SHAP), gradient based approaches such as integrated gradients, back propagation approaches such as DeepLIFT, model agnostic techniques such as Local Interpretable Model-agnostic Explanations (LIME), neural network and attention weight approaches such as Attention-Based Neural Network Models, or Deep Taylor Decomposition approaches such as Layer-wise Relevance Propagation (LRP) may be used to understand and interpret predictions made by the trained machine learning model.

At block 420, a set of candidate genes are identified from the ranked or otherwise sorted features that have the largest contribution or significant influence on the predicted residuals. Further, candidate genes are preferred to show high expression in relation to the target phenotype and low expression in the other correlated phenotype(s). One exemplary way in which candidate genes may be identified is with SHAP. Once ranked and sorted by their average absolute Shapley values, the highest ranking or sorted feature(s) (e.g., genes from the transcriptomic data) are identified as having the largest contribution or influence on the trained machine learning model output or prediction (e.g., residuals). The highest ranking or sorted feature(s) may be identified by sorting and identifying the feature(s) (e.g., a single gene, five genes, ten genes, fifteen genes, or the like) with the largest average of absolute Shapley values. The highest ranking or sorted feature(s) may be the candidate genes that can be tested in gene editing experiments to validate that they only impact the target phenotype and no other correlated phenotypes. Alternatively, the highest-ranking genes may also be filtered by their associated significant value (determined from permutation testing). Significance value may be determined using various statistical test statistics such as, and without limitation, p-value<cutoff, false discovery rate (FDR)<cutoff, or family-wise error rate (FWER)<cutoff, where cutoff is specified by a user. In some instances, the cutoff may be 0.05, 0.01, 0.001, 0.0001, etc.

### V. GENE DISCOVERY AND EDITING SYSTEM

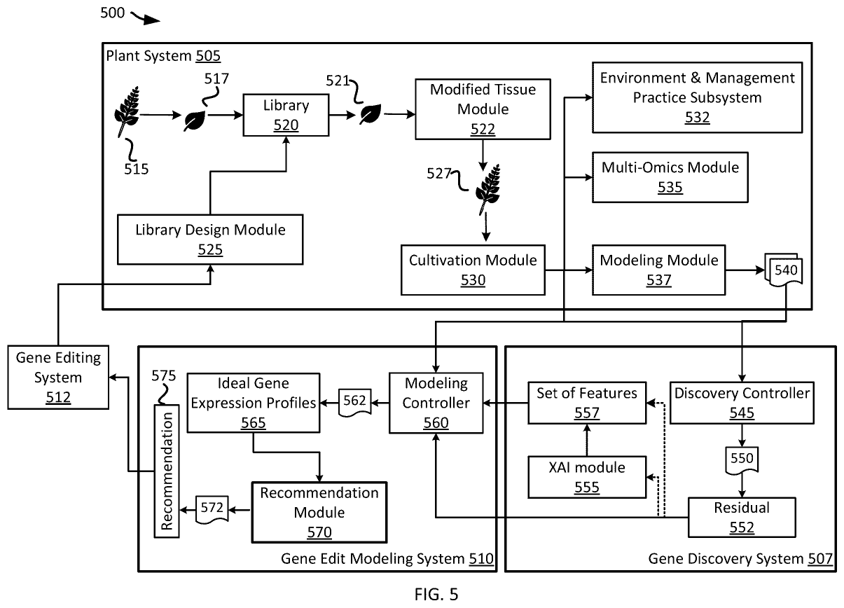

FIG. 5 shows a block diagram of a gene discovery and editing system 500. The gene discovery and editing system 500 is an example of a system implemented using computer programs on one or more computing devices in one or more locations in which the systems, components, and techniques described herein are implemented. The gene discovery and editing system 500 includes a plant system 505, a gene discovery system 507, a gene edit modeling system 510, and a gene editing system 512. Importantly, many of the systems and components described in the gene discovery and editing system 500 include systems and components described in the machine learning pipeline 200 in FIG. 2 and are detailed below.

The plant system 505 (e.g., the source of the samples used in FIG. 2) can execute a plant generation lifecycle by starting with a plant 515. The plant 515 can be dissected, either by an automated system, e.g., a robotic control system, or manually, to obtain plant tissue 517. The plant tissue 517 can then be modified using a library 520 to generate modified tissue 521. The library 520 is a collection of multiple versions of reagents, for example a collection of DNA sequences combinatorially assembled to give many different versions of a metabolic pathway. The library 520 can, for example, include plasmids, linear DNA fragments, synthetic guide RNAs (sgRNAs), RNAs, proteins, etc. A widely used library for plant gene modification is Agrobacterium tumefaciens. This bacterium is commonly used in plant biotechnology for the transfer of foreign genes into the genome of plants. The process involves using the Ti (tumor-inducing) plasmid of Agrobacterium, which contains the T-DNA (transfer DNA) region responsible for transferring genetic material into the plant cells. Various molecular biology techniques such as gene cloning, plasmid construction, agrobacterium-mediated transformation, CRISPR-Cas9 technology, and the like can be used alongside the library 520 such as Agrobacterium tumefaciens to modify plant genes. Regarding software or computational libraries, tools like CRISPR-Cas9 design software, sequence analysis tools, and genome editing platforms can be used in designing and analyzing gene modifications in plants. These tools assist in identifying target genes, designing guide RNAs, and predicting potential off-target effects, enhancing the efficiency and accuracy of gene editing in plants. The library 520 can be generated from a library design module 525 that compiles information for the library 520 from either output generated from a model during a previous life cycle of a plant, or from another source, for example, manual design from experts.

A modified tissue module 522 grows e.g., in a culture, the modified tissue 521 into a new plant 527, and provides the new plant 527 to a cultivation module 530. The cultivation module 530 may be governed by an environment and management practice subsystem 532 that dictates the environmental conditions and the management practices under which the new plant 527 is grown. As reference, the environmental and management factors described with respect to FIG. 2 are obtained from the environment and management practice subsystem 532. The cultivation module 530 obtains tissue samples and measurements from the new plant 527 as they grow, extracts data from the samples and measurements, and provides the extracted data to the environment and management practice subsystem 532 (e.g., phenotype measurements 224 from FIG. 2), the multi-omics module 535 (e.g., gene expression profiles 232 from FIG. 2), and/or the modeling module 537 (e.g., the prediction models 228, the machine learning algorithms 246, and the trained machine learning model 256 with respect to FIG. 2). The data extraction can include tissue sampling, molecule extraction and purification, and molecule quantification or identification, and can occur in any or a multitude of separate tissues/organs of the plant at various times of growth or continuously though-out the life cycle of the new plant 527. The environment and management practice subsystem 532 provides the extracted data (if received from the cultivation module 530), management practice profile data, and environment conditions profile data to the modeling module 537 for development of various models 540. The management practices profile data may include any adjustable aspect of the management of the growth of the new plant 527 at various times of growth or continuously though-out the life cycle of the new plant 527, (e.g., inputs such as fertilizer or water, the timing of planting, fertilizing, harvesting, etc.). The data environment conditions profile data may include the location-specific environmental conditions of the new plant 527 is exposed to at various times of growth or continuously though-out the life cycle of the new plant 527, (e.g., temperature, precipitation, soil properties, etc.). The multi-omics module 535 tracks the extracted data from the samples and measurements, generates multi-omics profiles (e.g., a gene expression profiles) of the small plant from the extracted data, and provides the multi-omics profiles to the modeling module 537 for development of the various models 540.

The modeling module 537 uses the received data (e.g., plant extracted data, multi-omics profiles, management practices profiles, environmental conditions profiles, etc.) for the development (e.g., design, training, validation, and deployment) of various models (e.g., machine learning models) that the gene discovery and editing system 500 can then use to guide growth of the new plant 527 and the generation of new plants with desired phenotypes. For example, the modeling module 537 can provide the trained or updated machine learning models to (i) the library designs module 525 to guide the modification of new plants, (ii) the environment and management practice subsystem 532 to guide the growth and management of the new plant 527, (iii) the gene discovery system 507 to generate phenotype predictions and facilitate gene discovery, and (iv) the gene edit modeling system 510 to model gene edits, generate ideal gene expression profiles, and facilitate the recommendation of gene edits.

The gene discovery system 507 includes a discovery controller 545 for obtaining input data (e.g., plant extracted data, multi-omics profiles such as gene expression profiles from multi-omics module 535, and environmental and management practices profiles from the environmental and management practice subsystem 532) for one or more plants (e.g., new plant 527 being grown in the plant system 505) and inputting the data into one or more models 550 (e.g., the machine learning model in the residual modeling subsystem 215 with respect to FIG. 2). The input data can be obtained from the environment and management practice subsystem 532, the cultivation module 530, the multi-omics module 535, and/or the modeling module 537. The one or more models 550 are constructed for the task of predicting a phenotype-specific residual 552 as the output data by learning relationships or correlations between features of the input data (e.g., gene expression profiles within the multi-omics profiles) and the residual. The one or more models 550 may be obtained from the modeling module 537 (various models 540). In some instances, a set of features 557 can be directly determined from the residuals 552 predicted from the model 550 because the relationships between the features of the input data are linear.

In some instances, the gene discovery system 507 requires an XAI module 555 (e.g., the XAI described in FIG. 4) for applying explainable techniques to the one or more models 550 to explain nonlinear relationships and obtain the importance of each feature for all predictions in a set of input data (e.g., a set of gene expression profiles). In some instances, the one or more models 550, which predicts the residuals 552, using gene expression profiles as inputs is inspected via XAI module 555 to identify features (e.g., one or more genes) that have the largest contribution or influence on the one or more models 550 output or prediction. The primary goal of the XAI module 555 is to define an importance measure (e.g., Shapley values) that identifies which features, such as gene(s), play an important role in the determination of a phenotype. The XAI module 555 outputs a set of features 557 that may be the candidate genes (described with respect to FIG. 3) to be involved in the molecular regulatory processes for that particular plant species and phenotype and are used in by the gene edit modeling system 510 for modeling gene edits.

The gene edit modeling system 510 includes a modeling controller 560 for obtaining the residuals 552 and the set of features 557 and inputting the residuals 552 and the set of feature(s) 557 into one or more models 562. The one or more models 562 may be obtained from the modeling module 537 (various models 540). The one or more models 562 use one or more various approaches (A)-(N) for modeling gene edits and generating ideal gene expression profiles 565. The ideal gene expression profiles 565 are a recommendation of gene expression for all genes in the set of features 557 for maximizing, minimizing, or otherwise modulating the residual 552. The gene edit modeling system 510 further includes a recommendation module 570 for comparing the ideal gene expression profiles 565 to a naturally occurring distribution of gene expression for the plant species (e.g., gene expressions within the multi-omics profiles) to determine a gene edit recommendation 575 that can be used by the gene editing system 512. The recommendation 575 may be for upregulating or down regulating a particular gene, a subgroup of genes, or each gene within the ideal gene expression profiles 565. In some instances, the recommendation module 570 uses one or more models 572 for determining where to make edits that will modulate the expression of genes based on the ideal gene expression profiles 565. These can be regions of multiple base pairs, potentially with strategies on how to make combinatorial edits to those regions, or exact locations with specific edits determined. The one or more models 572 may be a neural network or nonlinear model that predicts a target gene's expression level from a target gene's genomic context gathered from a genetically diverse plant population. The one or more models 572 may be trained on any of the following population data given the target gene's context: genomic sequence, SNPs, methylome, chromatin accessibility, and the like in combination with corresponding expression values. Recommendations for genomic edits may be extracted from the target gene's expression level following investigation of feature importance along with input feature ablation analysis of the one or more models 572.

The gene editing system 512 makes genetic edits or perturbations to the genome of a given plant species (e.g., new plant 527) in accordance with the recommendation 575. Examples of gene editing systems include CRISPR/Cas9, CRISPR/Cpf1, CRISPR/Cas12, CRISPR base editing, CRISPR inhibition, restriction enzymes, Zinc Finger Nucleases, Transcription activator-like effector nucleases (TALEN), and the like. For example, gene editing system 512 may make one or more combinatorial edits (“bashing”) in the gene regulatory genomic regions (promoters, 5′UTR, 3′UTR, terminator) of one or more target genes in order to modify their expression (upregulation or downregulation). Additionally or alternatively, gene editing system 512 may make one or more specific combinatorial edits to the binding site of a transcription factor of one or more target genes in order to modulate their effect on expression (upregulation or downregulation). Additionally or alternatively, gene editing system 512 may make one or more genomic modifications of any other region on the genome that may affect one or more target gene's expression (upregulation or downregulation) via genetic manipulation. Additionally or alternatively, gene editing system 512 may modulate the expression (upregulation or downregulation) of one or more target genes without genomic modifications, such as CRISPRi (target inhibition), CRISPRa (target activation), RNAi, etc. The system could also make crosses if the edits determined by system 510 are already accessible in the population. The modified genome of the given plant species may then be sent to the library design module 525 for use by the library 520 and the modified tissues module 522 to grow e.g., in a culture, modified tissues from the modified genome into a new plant.

### Examples

The following examples are offered by way of illustration, and not by way of limitation.

### VI. GENERATING RESIDUALS LABELS FOR TWO CORRELATED PHENOTYPES: INDOLE-GLUCOSINOLATE CONTENT AND FLOWERING TIME

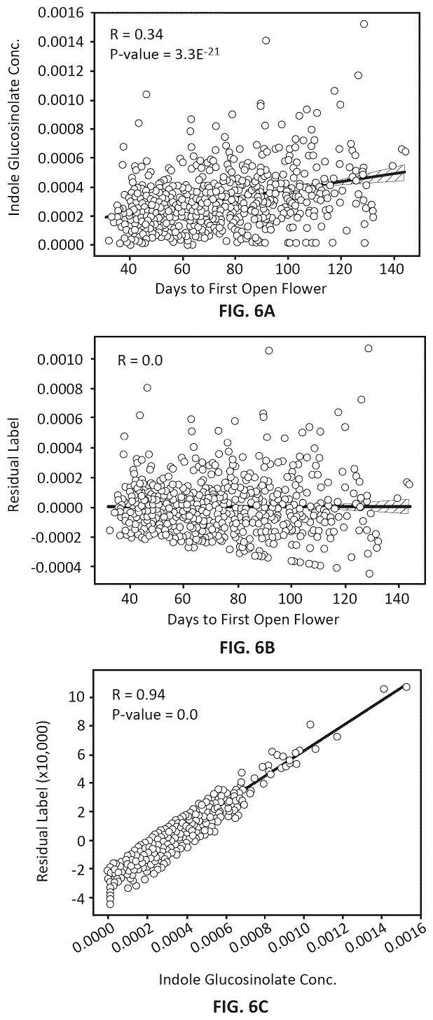

FIGS. 6A-6C shows scatter plots illustrating how phenotype correlation is used to generate residual labels. FIG. 6A shows the correlation between days to flower and indole glucosinolate content within the diversity panel of Arabidopsis thaliana. The correlation between flowering time and indole glucosinolate content is estimated at a Pearson R of 0.34. FIG. 6B shows the relationship between days to flower and the residuals as obtained from a linear regression model predicting indole glucosinolate concentration (dependent variable) from flowering time (independent variable). As illustrated, the correlation with flowering time is lost as flowering time was the independent variable regressed against in the linear regression. FIG. 6C shows that the relationship between the residuals and indole glucosinolate content is maintained. These residuals are used as the labels to predict from gene expression in downstream models.

### VII. ADDITIONAL CONSIDERATIONS

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium,” “storage” or “memory” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

What is claimed is:

1. A computer-implemented method comprising:
obtaining phenotype data and gene expression profiles for samples, wherein the phenotype data comprises measurements for correlated phenotypes, and wherein the correlated phenotypes comprise a target phenotype and one or more other phenotypes;
generating, using a prediction model, predicted measurements for the target phenotype for the samples based on relationships or correlations learned from the phenotype data for the one or more other phenotypes and the target phenotype;
determining residuals between the predicted measurements for the target phenotype and the obtained measurements for the target phenotype;
labeling, using the determined residuals, the gene expression profiles;
training, using the labeled gene expression profiles, a machine learning model to predict residuals from the labeled gene expression profiles; and
outputting the trained machine learning model.

2. The computer-implemented method of claim 1, wherein:
the prediction model is selected from a plurality of prediction models,
each prediction model in the plurality of prediction models is configured to model linear relationships, nonlinear relationships, or any combination thereof; and
the prediction model is configured to (i) model linear relationships and uses statistical functions to generate the predicted measurements for the target phenotype, (ii) model nonlinear relationships and uses statistical functions or machine learning models to generate the predicted measurements for the target phenotype, (iii) model both linear and nonlinear relationships and uses machine learning models to generate the predicted measurements for the target phenotype, or (iv) any combination of (i), (ii), and (iii).

3. The computer-implemented method of claim 1, wherein the training comprises: iterative operations to find a set of parameters for the machine learning model that minimizes a loss function for the machine learning model, wherein each iteration includes finding the set of parameters for the machine learning model so that a value of the loss function using the set of parameters is smaller than a value of the loss function using another set of parameters in a previous iteration, and wherein the loss function is configured to measure a difference in the predicted residuals and the determined residuals.

4. The computer-implemented method of claim 1, wherein the machine learning model is selected from a plurality of machine learning models, and wherein each machine learning model in the plurality of machine learning models can model either linear relationships, non-linear relationships, or any combination thereof.

5. The computer-implemented method of claim 1, wherein the machine learning model includes: (i) high interpretability and low accuracy that models linear relationships, (ii) low interpretability and high accuracy that models nonlinear relationships, (iii) high interpretability and high accuracy that models linear relationships, non-linear relationships, or any combination thereof, or (iv) any combination of (i), (ii), and (iii).

6. The computer-implemented method of claim 1, wherein the training comprises performing permutation testing, which comprises:
(a) shuffling the determined residuals in order to re-label the gene expression profiles;
(b) training the machine learning model, using the re-labeled gene expression profiles, to predict permuted residuals for the target phenotype;
(c) repeating (a) and (b) for a sufficient number of permutations to create an approximate test statistic null-distribution; and
(d) determining, based on the approximate test statistic null-distribution, statistical scores for each feature in the gene expression profiles.

7. A computer-implemented method comprising:
accessing a transcriptomic dataset for a set of correlated phenotypes comprising a target phenotype and one or more other phenotypes;
inputting the transcriptomic dataset into a machine learning model constructed for a task of predicting a residual that represents variation of the target phenotype that cannot be explained by the one or more other phenotypes;
generating, using the machine learning model, a predicted residual for the target phenotype based on the transcriptomic dataset;
analyzing decisions made by the machine learning model to predict the residual, wherein the analyzing comprises: generating (i) feature importance scores or (ii) statistical scores for features used in the prediction of the residual, and ranking or otherwise sorting the features based on the feature importance score or the statistical scores associated with each of the features;
identifying, a set of candidate genes for the target phenotype as having a largest contribution or influence on the residual based on the analyzing; and
identifying, based on the set of candidate genes, a set of genomic regions that when edited provides a requisite change in a gene expression profile to realize an expected phenotypic change.

8. The computer-implemented method of claim 7, wherein the transcriptomic dataset is collected from a sample, and wherein the transcriptomic dataset comprises expression data for all the genes in the sample or for a subset of genes in the sample.

9. The computer-implemented method of claim 7, wherein the machine learning model is configured to model linear relationships and wherein the feature importance scores are directly identified.

10. The computer-implemented method of claim 7, wherein the machine learning model utilizes one or more non-linear relationships to generate the predicted residual and wherein analyzing decisions comprises using an explainable artificial intelligence system and wherein the feature importance scores are indirectly identified using the explainable artificial intelligence system.

11. The computer-implemented method of claim 7, wherein the statistical scores are generated by performing permutation testing to create an approximate test statistic null-distribution.

12. A system comprising:
one or more data processors; and
a non-transitory computer readable medium storing instructions which, when executed on the one or more data processors, cause the one or more data processors to perform operations comprising:
obtaining phenotype data and gene expression profiles for samples, wherein:
the samples have correlated phenotypes,
the correlated phenotypes comprise one or more other phenotypes and a target phenotype, and
the phenotype data comprises measurements for the correlated phenotypes;
generating, using a prediction model, predicted measurements for the target phenotype based on relationships or correlations learned from the phenotype data for the one or more other phenotypes and the target phenotype;
determining residuals between the predicted measurements for the target phenotype and the obtained measurements for the target phenotype;
labeling, using the determined residuals, the gene expression profiles;
training, using the labeled gene expression profiles, a machine learning model to predict residuals from the labeled gene expression profiles; and
outputting the trained machine learning model.

13. The system of claim 12, wherein:
the prediction model is selected from a plurality of prediction models,
each prediction model in the plurality of prediction models is configured to model linear relationships, nonlinear relationships, or any combination thereof; and
the prediction model is configured to (i) model linear relationships and uses statistical functions to generate the predicted measurements for the target phenotype, (ii) model nonlinear relationships and uses statistical functions or machine learning models to generate the predicted measurements for the target phenotype, (iii) model both linear and nonlinear relationships and uses machine learning models to generate the measurements for the predicted target phenotype, or (iv) any combination of (i), (ii), and (iii).

14. The system of claim 12, wherein the training comprises: iterative operations to find a set of parameters for the machine learning model that minimizes a loss function for the machine learning model, wherein each iteration includes finding the set of parameters for the machine learning model so that a value of the loss function using the set of parameters is smaller than a value of the loss function using another set of parameters in a previous iteration, and wherein the loss function is configured to measure a difference in the predicted residuals and the determined residuals.

15. The system of claim 14, wherein the machine learning model is selected from a plurality of machine learning models, and wherein each machine learning model in the plurality of machine learning models can model either linear relationships or non-linear relationships, or any combination thereof.

16. The system of claim 15, wherein the machine learning model includes: (i) high interpretability and low accuracy that models linear relationships, (ii) low interpretability and high accuracy that models nonlinear relationships, (iii) high interpretability and high accuracy that models linear relationships, non-linear relationships, or any combination thereof, or (iv) any combination of (i), (ii), and (iii).

17. The system of claim 12, wherein the training comprises performing permutation testing, which comprises:
(a) shuffling the determined residuals in order to re-label the gene expression profiles;
(b) training the machine learning model, using the re-labeled gene expression profiles, to predict permuted residuals for the target phenotype;
(c) repeating (a) and (b) for a sufficient number of permutations to create an approximate test statistic null-distribution; and
(d) determining, based on the approximate test statistic null-distribution, statistical scores for each feature in the gene expression profiles.

18. The system of claim 17, wherein the training further comprises:
ranking the features based on the statistical scores; and
identifying a set of candidate genes for the target phenotype based on the ranking, wherein the outputting further comprises outputting the set of candidate genes.

19. The system of claim 18, wherein the operations further comprise:
obtaining a transcriptomic dataset for a test sample;
inputting the transcriptomic dataset to the trained machine learning model; and
generating a predicted residual and a set of genes to be edited for the target phenotype using the trained machine learning model based on the transcriptomic dataset.

20. The system of claim 19, further comprising a gene editing subsystem, wherein the gene editing subsystem is configured to perform gene editing on the set of genes of the test sample.
